(19) United States
(12) Patent Application Publication
(10) Pub. No.: US 2026/0267834 A1
(43) Pub. Date: Sep. 10, 2026

(54) INFORMATION PROCESSING DEVICE, INFORMATION PROCESSING METHOD, COMPUTER PROGRAM AND NON-TRANSITORY COMPUTER-READABLE MEDIUM

(71) Applicant: NIHON KOHDEN CORPORATION, Tokyo (JP)

(72) Inventors: Takayuki SUGIYAMA, Saitama (JP); Akihiko Yoshimura, Saitama (JP)

(73) Assignee: NIHON KOHDEN CORPORATION, Tokyo (JP)

(21) Appl. No.: 19/549,728

(22) Filed: Feb. 25, 2026

(30) Foreign Application Priority Data
Mar. 7, 2025 (JP) 2025-036216

(51) Int. Cl. G06F 16/215; G06F 16/26

(52) U.S. Cl. CPC G06F 16/215; G06F 16/26

(57) ABSTRACT

An information processing device includes one or more processors, and one or more memories configured to store at least one instruction configured to be executed by the one or more processors. The information processing device is configured to acquire first physiological information of a subject, and second physiological information of the subject, the second physiological information being different from the first physiological information. The information processing device is configured to calculate third physiological information, based on the first physiological information, the second physiological information, and a predetermined calculation formula stored in advance in the one or more memories.

### CROSS-REFERENCE TO RELATED APPLICATIONS

This application is based on and claims priority under 35 USC 119 from Japanese Patent Application No. 2025-036216 filed on Mar. 7, 2025, the entire content of which is incorporated herein by reference.

### TECHNICAL FIELD

The presently disclosed subject matter relates to an information processing device, an information processing method, a computer program, and a non-transitory computer-readable medium.

### BACKGROUND ART

JP2003-339655A discloses a physiological information measurement device including a sensor attachment state detection unit that detects an attachment state of a sensor to a subject, and a notification unit that issues a notification prompting an input of subject information based on the attachment state of the sensor.

A medical worker such as a doctor may use not only primary physiological information directly acquired from a subject but also secondary physiological information calculated based on the primary physiological information and a predetermined calculation formula, for example, when confirming a condition of the subject. The primary physiological information is, for example, a continuous cardiac output (CCO), height information, weight information, or the like. The primary physiological information is acquired by an information processing device such as a bedside monitor by receiving the information from an external device or by being input into the information processing device. The secondary physiological information is, for example, a continuous cardiac index (CCI). For example, when the bedside monitor calculates the continuous cardiac output, the bedside monitor cannot calculate the continuous cardiac index unless all the physiological information (the continuous cardiac output, the height information, and the weight information) necessary for a calculation formula used for calculating the continuous cardiac index is acquired. Therefore, for example, even if the bedside monitor can acquire the continuous cardiac output and the height information, the bedside monitor cannot calculate the continuous cardiac index unless the weight information is acquired. In the information processing device of the related art, although it is possible to alert a medical worker or the like to input the primary physiological information, it is not sufficiently considered to alert the medical worker or the like that the secondary physiological information cannot be calculated due to the absence of the primary physiological information. In this respect, there is room for improvement in the information processing device of the related art.

### SUMMARY OF INVENTION

Aspect of non-limiting embodiments of the present disclosure relates to provide an information processing device, an information processing method, a computer program, and a non-transitory computer-readable medium capable of reducing occurrence of a situation in which secondary physiological information calculated by primary physiological information and a predetermined calculation formula cannot be calculated due to absence of the primary physiological information.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided an information processing device including:
one or more processors; and
one or more memories configured to store at least one instruction configured to be executed by the one or more processors,
in which the information processing device is configured to acquire:
   first physiological information of a subject; and
   second physiological information of the subject, the second physiological information being different from the first physiological information,
the information processing device is configured to calculate third physiological information, based on the first physiological information, the second physiological information, and a predetermined calculation formula stored in advance in the one or more memories, and
when the at least one instruction is executed by the one or more processors,
in a case where there is the first physiological information and/or the second physiological information that have not been acquired by the information processing device in calculating the third physiological information,
the information processing device generates missing information indicating that there is the first physiological information and/or the second physiological information that have not been acquired by the information processing device among the first physiological information and the second physiological information necessary for calculating the third physiological information.

### BRIEF DESCRIPTION OF DRAWINGS

Exemplary embodiment(s) of the present invention will be described in detail based on the following figures, wherein:

FIG. 1 is a system configuration diagram of an information processing system according to a first embodiment;

FIG. 2 is a flowchart illustrating an information processing method performed in the present embodiment;

FIG. 3 illustrates display data displayed on a display; and

FIG. 4 illustrates a correspondence relationship table.

### DESCRIPTION OF EMBODIMENTS

Hereinafter, examples of an embodiments of the presently disclosed subject matter will be described with reference to the drawings.

### Overall Configuration

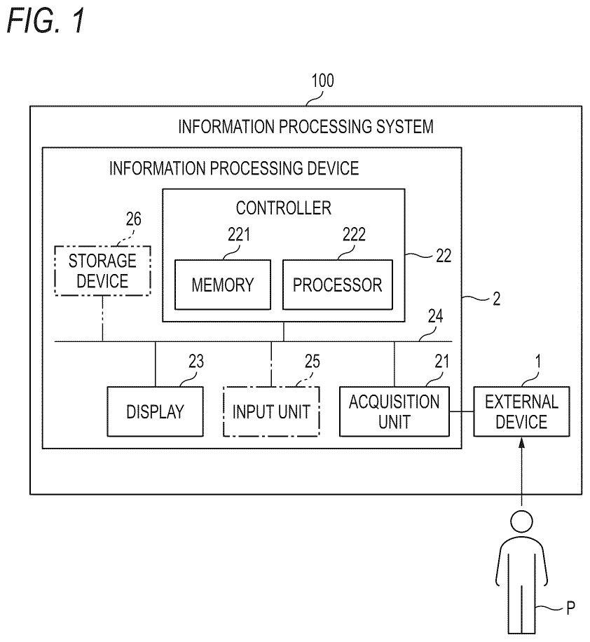

First, an information processing system 100 will be described with reference to FIG. 1. The information processing system 100 is used in, for example, a medical facility such as a hospital ward. As illustrated in FIG. 1, the information processing system 100 can include an external device 1 and an information processing device 2. The external device 1 is communicably connected to the information processing device 2 in a wired or wireless manner.

The external device 1 is, for example, a device used for evaluating circulatory dynamics. For example, the external device 1 is configured to generate first physiological information such as a cardiac output (CO), a continuous cardiac output (CCO), a mean pulmonary arterial pressure (mPAP), and a pulmonary capillary wedge pressure (PCWP), based on an arterial blood pressure waveform of a subject P measured by the external device 1. The external device 1 is configured to transmit the generated first physiological information to the information processing device 2. In the present embodiment, the external device 1 in a state of being connected to the information processing device 2 is configured to automatically transmit the first physiological information to the information processing device 2.

The external device 1 is configured such that the second physiological information is input by a user of the external device 1 (a medical worker or the like who operates the external device 1). The second physiological information is, for example, height information indicating the height of the subject P, weight information indicating the weight of the subject P, or the like. As described above, the “physiological information” in the present specification can also include physical information such as the height information and the weight information. The external device 1 is configured to acquire the second physiological information, for example, as the second physiological information is input to the external device 1 by the user of the external device 1. The external device 1 is configured to transmit the acquired second physiological information to the information processing device 2.

The information processing device 2 is, for example, a bedside monitor. The information processing device 2 can include an acquisition unit 21, a controller 22, and a display 23. These components are communicably connected to each other via a bus 24.

The acquisition unit 21 is configured to acquire the first physiological information and/or the second physiological information, as the information processing device 2 receives the first physiological information and/or the second physiological information from the external device 1. Therefore, the information processing device 2 in a state of being connected to the external device 1 is configured to automatically acquire the first physiological information and/or the second physiological information from the external device 1.

The controller 22 can include a memory 221 and a processor 222, as a hardware configuration. For example, the memory 221 can include a read only memory (ROM) configured to store various computer programs, and a random access memory (RAM) having a plurality of work areas in which various computer programs to be executed by the processor 222 are stored. The processor 222 is, for example, a central processing unit (CPU), and is configured to load a computer program specified from various computer programs incorporated in the ROM on the RAM and execute various processing in cooperation with the RAM. In the present embodiment, the memory 221 is configured to store, for example, a predetermined calculation formula for calculating third physiological information to be described later. Details of the predetermined calculation formula will be described later. The predetermined calculation formula may be stored in the memory 221 as a part of the computer program.

In the present embodiment, a computer-readable medium may be used. The computer-readable medium refers to a type of physical memory (RAM, ROM, or the like) that may store information and data readable by the processor. The computer-readable medium may store instructions related to execution processing by one or more processors. The term “computer-readable medium” can include a tangible item and excludes a carrier wave or a temporary signal (that is, refers to a non-transitory medium). Examples of the non-transitory computer-readable medium can include a magnetic recording medium (for example, a flexible disk, a magnetic tape, or a hard disk drive), a magneto-optical recording medium (for example, a magneto-optical disk), a CD read only memory (CD-ROM), a CD-R, a CD-R/W, and a semiconductor memory (for example, a mask ROM, a programmable ROM (PROM), an erasable PROM (EPROM), or a flash ROM).

The controller 22 is configured to determine whether the external device 1 and the information processing device 2 are connected.

The controller 22 is configured to calculate the third physiological information, based on the first physiological information, the second physiological information, and the predetermined calculation formula stored in advance in the memory 221. The third physiological information is, for example, a continuous cardiac index (CCI), a pulmonary vascular resistance index (PVRI), or the like. The continuous cardiac index is used as an index for evaluating a cardiac function of the subject P, and the pulmonary vascular resistance index is used as an index for evaluating a pulmonary function of the subject P.

The continuous cardiac index is obtained based on the continuous cardiac output, a body surface area (BSA), and the following formula (1).

$$The continuous cardiac index = the continuous cardiac output / the body surface area \qquad ( 1 )$$

The body surface area can be obtained by the height information, the weight information, and the following formula (2).

$$The body surface area ( BSA ) = the {height}^{0.725} ( m ) * the {weight}^{0.425} ( kg ) * 0.007184 \qquad ( 2 )$$

The pulmonary vascular resistance index is obtained based on the mean pulmonary arterial pressure, the pulmonary capillary wedge pressure, the cardiac index (CI), and the following formula (3).

$$The pulmonary vascular resistance index = ( the mean pulmonary arterial pressure - the pulmonary capillary wedge pressure ) / the cardiac index \qquad ( 3 )$$

The cardiac index can be obtained by the cardiac output, the body surface area, and the following formula (4).

$$The cardiac index = the cardiac output / the body surface area \qquad ( 4 )$$

Since the continuous cardiac output and the cardiac output are indices that are greatly affected by physique of each person, the cardiac function and the pulmonary function of the subject P can be more appropriately evaluated by using the continuous cardiac index and the pulmonary vascular resistance index (that is, values in consideration of the body surface area).

When calculating the third physiological information, the controller 22 is configured to specify the first physiological information and the second physiological information necessary for calculating the third physiological information. For example, in a case where the controller 22 is set to calculate the continuous cardiac index, the controller 22 is configured to specify the continuous cardiac output as the first physiological information necessary for calculating the continuous cardiac index, and specify the height information and the weight information as the second physiological information necessary for calculating the continuous cardiac index.

In a case where the first physiological information and the second physiological information necessary for calculating the third physiological information are specified, the controller 22 is configured to determine whether all the specified first physiological information and second physiological information have been acquired. In a case where all the first physiological information and the second physiological information necessary for calculating the third physiological information have been acquired, the controller 22 is configured to calculate the third physiological information based on the first physiological information and the second physiological information. On the other hand, in a case where the first physiological information and/or the second physiological information necessary for calculating the third physiological information has not been acquired, the controller 22 generates missing information. Note that the missing information is information indicating that there is the first physiological information and/or the second physiological information that has not been acquired by the information processing device 2 among the first physiological information and the second physiological information necessary for calculating the third physiological information.

Based on the missing information, the controller 22 is configured to generate missing physiological type information indicating the type of missing first physiological information and/or second physiological information. In addition, based on the missing information, the controller 22 is configured to generate uncalculated physiological type information indicating third physiological information that cannot be calculated because the first physiological information and/or the second physiological information is missing.

The controller 22 is configured to generate display data including the missing information, the missing physiological type information, and the uncalculated physiological type information. The controller 22 is configured to output the generated display data to the display 23.

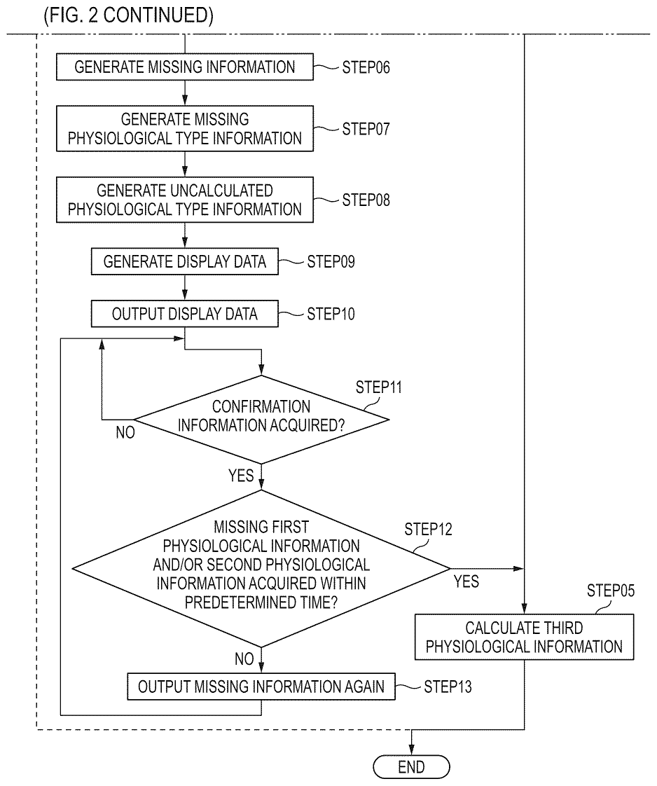

After outputting the display data to the display 23, the controller 22 is configured to determine whether confirmation information indicating that the user has confirmed the missing information has been acquired. The controller 22 is configured to acquire the confirmation information, for example, in a case where the user (the medical worker or the like) of the information processing device 2 clicks a confirmation button B1 (see FIG. 3) displayed on the display 23. However, a method for acquiring the confirmation information is not limited to this example. For example, the controller 22 may be configured to acquire the confirmation information by inputting, to the information processing device 2, a message indicating that the user of the information processing device 2 has confirmed the missing information.

After outputting the display data to the display 23, the controller 22 is configured to output the display data including the missing information and the like a predetermined number of times until the confirmation information is acquired. That is, the controller 22 is configured to output the missing information the predetermined number of times until the confirmation information is acquired after the missing information is output. Note that in the present embodiment, the predetermined number of times is once. However, the controller 22 may be configured to output the missing information twice or more until the confirmation information is acquired after the missing information is output.

The controller 22 is configured to determine whether the missing first physiological information and/or second physiological information has been acquired within a predetermined time (for example, 24 hours) after the information processing device 2 acquires the confirmation information. In a case where the missing first physiological information and/or the second physiological information is acquired within the predetermined time after the information processing device 2 acquires the confirmation information, the controller 22 is configured to calculate the third physiological information based on the first physiological information and the second physiological information. On the other hand, in a case where the missing first physiological information and/or second physiological information is not acquired within the predetermined time after the information processing device 2 acquires the confirmation information, the controller 22 outputs the display data including the missing information and the like to the display 23 again.

The display 23 is configured to display the display data output from the controller 22. The display 23 is, for example, a touch screen display such as a liquid crystal display or an organic EL display.

### First Embodiment

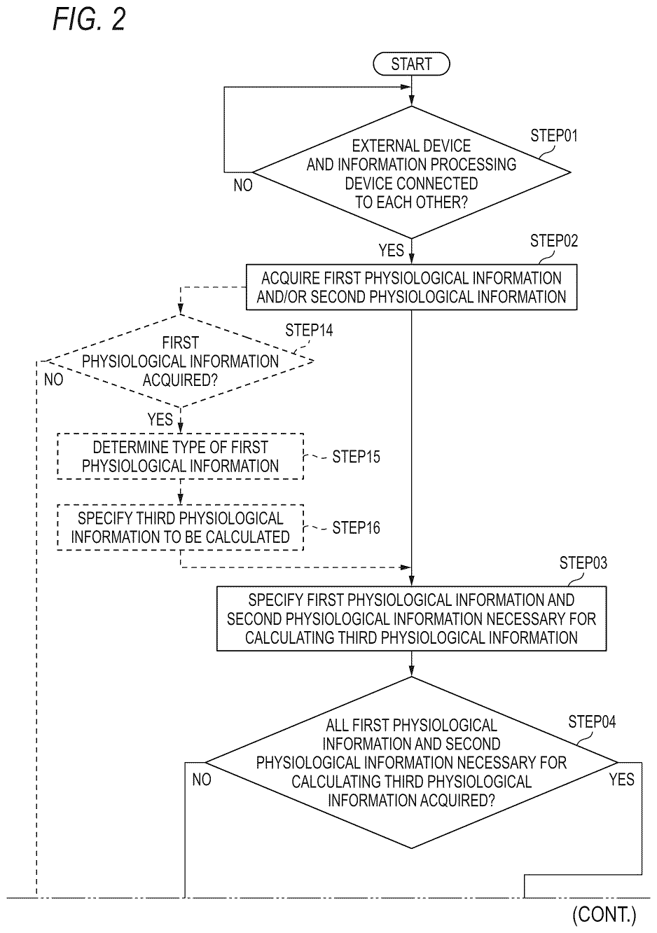

Next, an information processing method performed in a first embodiment will be described with reference to FIG. 2. FIG. 2 is a flowchart illustrating the information processing method performed in the present embodiment. In the present embodiment, the information processing device 2 is set in advance such that the controller 22 is configured to calculate the continuous cardiac index and the pulmonary vascular resistance index. In the present embodiment, it is assumed that the external device 1 has acquired the continuous cardiac output, the cardiac output, the mean pulmonary arterial pressure, the pulmonary capillary wedge pressure, and the height information before the information processing method is performed, but has not acquired the weight information.

The controller 22 of the information processing device 2 determines whether the external device 1 and the information processing device 2 are connected (STEP01). In a case where the external device 1 and the information processing device 2 are connected (YES in STEP01), the external device 1 transmits the first physiological information and/or the second physiological information to the information processing device 2. Therefore, in this case, the acquisition unit 21 automatically acquires the first physiological information and the second physiological information from the external device 1 as long as a connection state between the external device 1 and the information processing device 2 is maintained (STEP02). On the other hand, in a case where the external device 1 and the information processing device 2 are not connected (NO in STEP01), the controller 22 performs STEP01 again. In the present embodiment, in a case where the external device 1 and the information processing device 2 are connected (YES in STEP01), the acquisition unit 21 acquires the continuous cardiac output, the cardiac output, the mean pulmonary arterial pressure, the pulmonary capillary wedge pressure, and the height information, from the external device 1 (STEP02).

The controller 22 specifies the first physiological information and the second physiological information necessary for calculating the third physiological information (STEP03). In the present embodiment, since the information processing device 2 calculates the continuous cardiac index and the pulmonary vascular resistance index, the controller 22 specifies the continuous cardiac output, the cardiac output, the mean pulmonary arterial pressure, and the pulmonary capillary wedge pressure as the first physiological information necessary for calculating the continuous cardiac index and the pulmonary vascular resistance index, and specifies the height information and the weight information as the second physiological information (STEP03).

The controller 22 determines whether all the first physiological information and the second physiological information necessary for calculating the third physiological information have been acquired (STEP04). When it is determined that all the first physiological information and the second physiological information necessary for calculating the third physiological information have been acquired (YES in STEP04), the controller 22 calculates the third physiological information based on the acquired first physiological information and second physiological information (STEP05). On the other hand, when it is determined that the first physiological information and/or the second physiological information necessary for calculating the third physiological information has not been acquired (NO in STEP04), the controller 22 generates the missing information based on the determination (STEP06). In the present embodiment, the acquisition unit 21 does not acquire the weight information. Therefore, the controller 22 determines that the weight information necessary for calculating the continuous cardiac index and the pulmonary vascular resistance index has not been acquired (NO in STEP04), and generates the missing information based on the determination (STEP06). Note that the missing information generated in the present embodiment is information indicating that the information processing device 2 has not acquired the weight information among the first physiological information and the second physiological information specified in STEP03.

Based on the missing information, the controller 22 generates the missing physiological type information indicating the type of the missing first physiological information and/or second physiological information (STEP07). In the present embodiment, since the weight information is missing, the controller 22 generates the missing physiological type information indicating that the missing second physiological information is the weight information (STEP07).

Based on the missing information, the controller 22 generates the uncalculated physiological type information indicating a type of the third physiological information that cannot be calculated due to absence of the first physiological information and/or the second physiological information (STEP08). In the present embodiment, since the continuous cardiac index and the pulmonary vascular resistance index cannot be calculated due to the absence of the weight information, the controller 22 generates the uncalculated physiological type information indicating that the third physiological information that cannot be calculated is the continuous cardiac index and the pulmonary vascular resistance index (STEP08).

The controller 22 generates the display data including the missing information, the missing physiological type information, and the uncalculated physiological type information (STEP09). The controller 22 transmits the generated display data to the display 23 (STEP10). In this way, the missing information, the missing physiological type information, and the uncalculated physiological type information are output. In the present embodiment, when STEP10 is performed, display data illustrated in FIG. 3 is displayed on the display 23. In the present embodiment, the controller 22 is configured to output the display data including the missing information and the like only once until the confirmation information is acquired after outputting the display data to the display 23.

As illustrated in FIG. 3, the display data can include a first message M1, a second message M2, and a third message M3. The first message M1 is a message indicating that the weight information is missing. The second message M2 is a message indicating the type (the continuous cardiac index and the pulmonary vascular resistance index) of the third physiological information that cannot be calculated due to the absence of the weight information. The third message M3 is a message indicating that the weight information is required to calculate the continuous cardiac index and the pulmonary vascular resistance index (that is, the missing second physiological information is the weight information). Therefore, by visually recognizing the display data illustrated in FIG. 3, the user can recognize the missing second physiological information (the weight information), the third physiological information (the continuous cardiac index and the pulmonary vascular resistance index) that cannot be calculated due to the absence, and the second physiological information (the weight information) necessary for calculating the third physiological information that cannot be calculated.

Returning to FIG. 2, STEP11 will be described. After outputting the display data including the missing information and the like, the controller 22 determines whether the confirmation information indicating that the user has confirmed the missing information has been acquired (STEP11). When it is determined that the confirmation information has been acquired (YES in STEP11), the controller 22 performs STEP12. On the other hand, when it is determined that the confirmation information has not been acquired (NO in STEP11), the controller 22 performs STEP11 again. In the present embodiment, the controller 22 acquires the confirmation information by the medical worker who operates the information processing device 2 clicking the confirmation button B1 (see FIG. 3) displayed on the display 23.

As illustrated in FIG. 2, the controller 22 determines whether the missing first physiological information and/or second physiological information has been acquired within the predetermined time (24 hours in the present embodiment) after the information processing device 2 acquires the confirmation information (STEP12). When it is determined that the missing first physiological information and/or second physiological information has been acquired within the predetermined time after the information processing device 2 acquires the confirmation information (YES in STEP12), the controller 22 calculates the third physiological information based on the acquired first physiological information and second physiological information (STEP05). On the other hand, when it is determined that the missing first physiological information and/or second physiological information has not been acquired within the predetermined time after the information processing device 2 acquires the confirmation information (NO in STEP12), the controller 22 outputs the missing information again (STEP13). In the present embodiment, the controller 22 is configured to output the missing information again by outputting the display data including the missing information to the display 23 again. In addition, in the present embodiment, when the acquisition unit 21 acquires the weight information within 24 hours after the information processing device 2 acquires the confirmation information (YES in STEP12), the controller 22 calculates the body surface area based on the height information, the weight information, and the above formula (2). Next, the controller 22 calculates the continuous cardiac index, based on the continuous cardiac output, the body surface area, and the above formula (1) (STEP05). Further, the controller 22 calculates the cardiac index, based on the cardiac output, the body surface area, and the above formula (4). Next, the controller 22 calculates the pulmonary vascular resistance index, based on the cardiac index, the mean pulmonary arterial pressure, the pulmonary capillary wedge pressure, and the above formula (3) (STEP05). Since the continuous cardiac index and the pulmonary vascular resistance index are displayed on the display 23, for example, a user such as a medical worker can confirm the continuous cardiac index and the pulmonary vascular resistance index by visually recognizing the display 23.

In the first embodiment, according to the information processing device 2, in a case where there is the first physiological information and/or the second physiological information that has not been acquired by the acquisition unit 21 in calculating the third physiological information, the controller 22 is configured to generate the missing information indicating that there is the first physiological information and/or the second physiological information that has not been acquired by the acquisition unit 21 among the first physiological information and the second physiological information necessary for calculating the third physiological information. Therefore, by using such missing information, the user (the medical worker or the like) of the information processing device 2 recognizes that there is the first physiological information and/or the second physiological information that has not been acquired by the information processing device 2 among the first physiological information and the second physiological information necessary for calculating the third physiological information. After confirming the missing information, the user of the information processing device 2 takes necessary measures for the information processing device 2 to acquire the first physiological information and/or the second physiological information that has not been acquired by the acquisition unit 21. Accordingly, when the information processing device 2 acquires the missing first physiological information and/or second physiological information, the controller 22 calculates the third physiological information based on the first physiological information, the second physiological information, and the predetermined calculation formula stored in advance in the information processing device 2. As described above, according to the information processing device 2, it is possible to reduce occurrence of a situation in which the secondary physiological information (the third physiological information) calculated by the primary physiological information (the first physiological information and the second physiological information) and the predetermined calculation formula cannot be calculated due to absence of the primary physiological information. In addition, the information processing method, the computer program and the non-transitory computer-readable medium according to the above configurations can also achieve the same or similar effects.

In the first embodiment, the information processing device 2 is configured to acquire the first physiological information and the second physiological information by receiving the first physiological information and the second physiological information from the external device 1 configured to communicate with the information processing device 2. The first physiological information is generated by the external device 1, and the second physiological information is input to the external device 1 by the user of the external device 1. Therefore, regarding the first physiological information, if the information processing device 2 and the external device 1 are in a communicable state, the information processing device 2 can basically appropriately acquire the first physiological information. On the other hand, regarding the second physiological information, for example, the information processing device 2 cannot acquire the second physiological information unless the user of the external device 1 inputs the second physiological information to the external device 1. According to the information processing device 2, it is possible to prevent the occurrence of the situation in which the third physiological information cannot be calculated due to non-acquisition of the second physiological information acquired by being input to the external device 1. That is, according to the information processing device 2, it is possible to more effectively prevent the occurrence of the situation in which the third physiological information cannot be calculated. Further, since the information processing device 2 can acquire the first physiological information and the second physiological information only by receiving the first physiological information and the second physiological information from the external device 1, the configuration of the information processing device 2 can be simplified.

In the first embodiment, since the information processing device 2 outputs the missing information only once until the confirmation information indicating that the user of the information processing device 2 has confirmed the missing information is acquired, the user is not bothered, for example, as compared with a case where the missing information is constantly output.

In the first embodiment, according to the information processing device 2, when it is determined that there is the first physiological information and/or the second physiological information that has not been acquired by the acquisition unit 21 among the first physiological information and/or the second physiological information specified as the first physiological information and/or the second physiological information necessary for calculating the third physiological information when 24 hours have elapsed after the confirmation information is acquired, the controller 22 outputs the missing information again. Therefore, for example, when the user confirms the missing information but does not input the first physiological information and/or the second physiological information that has not been acquired by the acquisition unit 21 to the information processing device 2, it is possible to prevent the second physiological information has not been acquired by the acquisition unit 21 from being left unentered.

In the first embodiment, since the information processing device 2 generates the display data including the missing information, the user of the information processing device 2 can visually recognize the missing information. Therefore, according to the information processing device 2, the user can relatively easily recognize the missing information.

In the first embodiment, the information processing device 2 generates the display data including the missing physiological type information indicating the missing first physiological information and/or second physiological information. By using such display data, the user can recognize the second physiological information that has not been acquired by the acquisition unit 21, and can easily estimate the third physiological information that has not been calculated by the controller 22.

In the first embodiment, the information processing device 2 generates the display data including the uncalculated physiological type information indicating the third physiological information that cannot be calculated because the first physiological information and/or the second physiological information is missing. By using such display data, the user can recognize the third physiological information that cannot be calculated by the controller 22, and can easily estimate the first physiological information and/or the second physiological information that have not been acquired by the acquisition unit 21.

### Second Embodiment

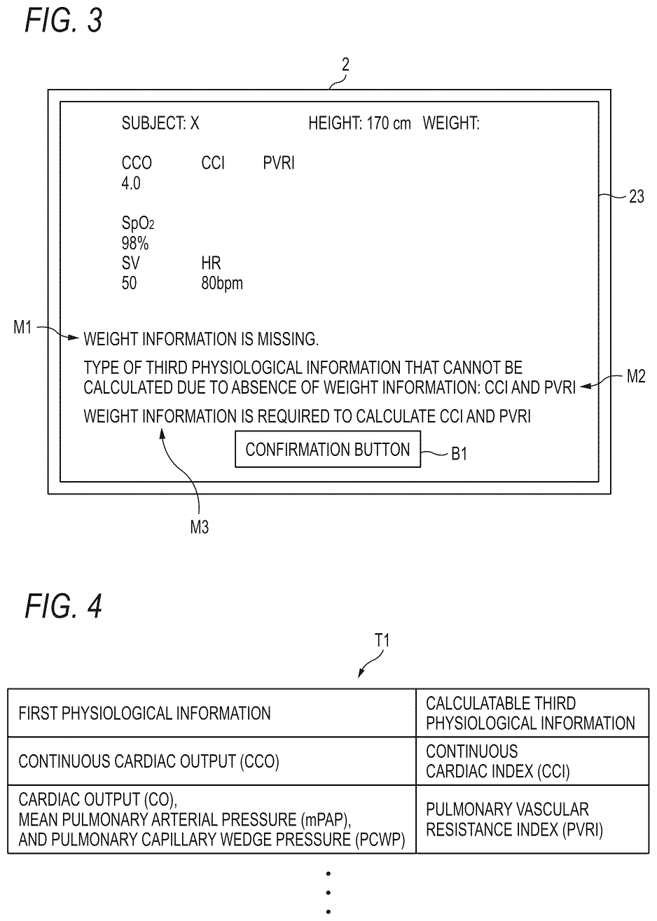

Next, an information processing method performed in a second embodiment will be described with reference to FIGS. 2 and 4. FIG. 4 illustrates a correspondence relationship table T1. In the second embodiment, parts the same or similar to those of the first embodiment are denoted by the same or similar reference numerals as those of the first embodiment, and description of repeated parts is omitted as appropriate.

In the present embodiment, the memory 221 is configured to store a correspondence relationship table T1 indicating a correspondence relationship between the first physiological information and calculatable third physiological information using the first physiological information. As illustrated in FIG. 4, for example, the calculatable third physiological information when the first physiological information is the continuous cardiac output is a continuous cardiac index.

In the present embodiment, the controller 22 is configured to determine the type of the first physiological information acquired by the acquisition unit 21, and specify the third physiological information to be calculated according to the type of the first physiological information. More specifically, the controller 22 is configured to specify the third physiological information to be calculated, based on the first physiological information acquired by the acquisition unit 21 and the correspondence relationship table T1.

As described above, the present embodiment is different from the first embodiment in that the correspondence relationship table T1 is stored in the memory 221 and the controller 22 determines the type of the first physiological information and specifies the third physiological information to be calculated according to the type of the first physiological information. Therefore, in the present embodiment, as indicated by a broken line in FIG. 2, the controller 22 performs STEP02, performs STEP14 to STEP16, and then performs STEP03. In the present embodiment, since the third physiological information calculated by the controller 22 is determined according to the type of the first physiological information acquired by the acquisition unit 21, the third physiological information calculated by the controller 22 may not be set in advance.

STEP01 and STEP02 are the same as or similar to those in the first embodiment.

The controller 22 determines whether the first physiological information has been acquired from the external device 1 (STEP14). When the controller 22 determines that the first physiological information has been acquired from the external device 1 (YES in STEP14), the controller 22 determines the type of the first physiological information (STEP15). On the other hand, when it is determined that the first physiological information has not been acquired from the external device 1 (NO in STEP14), the controller 22 ends the information processing. In the present embodiment, since the acquisition unit 21 acquires the continuous cardiac output, the cardiac output, the mean pulmonary arterial pressure, and the pulmonary capillary wedge pressure from the external device 1 as the first physiological information (YES in STEP14), the controller 22 determines that the types of the first physiological information are the continuous cardiac output, the cardiac output, the mean pulmonary arterial pressure, and the pulmonary capillary wedge pressure (STEP15).

The controller 22 specifies the third physiological information to be calculated according to the type of the first physiological information (STEP16). In the present embodiment, since the types of the first physiological information are the continuous cardiac output, the cardiac output, the mean pulmonary arterial pressure, and the pulmonary capillary wedge pressure, the controller 22 specifies the continuous cardiac index and the pulmonary vascular resistance index as the third physiological information to be calculated (STEP16). STEP03 to STEP13 are the same as or similar to those in the first embodiment.

Also in the present embodiment, the information processing device 2 has effects the same as or similar to those in the first embodiment.

In the second embodiment, the information processing device 2 specifies the third physiological information to be calculated by the controller 22 according to the type of the first physiological information acquired by the acquisition unit 21. Therefore, the information processing device 2 may specify the first physiological information and the second physiological information necessary for calculating the specified third physiological information, and determine whether there is the first physiological information and/or the second physiological information that has not been acquired by the acquisition unit 21 among the specified first physiological information and second physiological information. That is, the information processing device 2 only needs to determine whether the first physiological information and the second physiological information necessary for calculating the specified third physiological information are missing. Therefore, according to the information processing device 2, it is possible to reduce a load of the information processing performed by the controller 22.

The above embodiments are intended to facilitate understanding of the presently disclosed subject matter, and are not intended to limit the presently disclosed subject matter. The presently disclosed subject matter may be modified or improved without departing from the gist thereof.

In the above embodiments, the information processing device 2 may include an input unit 25 as indicated by a one-dot chain line in FIG. 1. In this case, the input unit 25 acquires the second physiological information when the second physiological information is input to the input unit 25 by a person (for example, the user such as a medical worker) who operates the information processing device 2. Therefore, in this case, the information processing device 2 can also acquire the second physiological information by the user such as a medical worker inputting the second physiological information to the input unit 25. The input unit 25 is a touch panel superimposed on the display 23, an operation button attached to a housing of the information processing device 2, and the like.

In the above embodiments, the information processing device 2 may include a storage device 26 (an example of a memory) as indicated by a two-dot chain line in FIG. 1. In this case, the storage device 26 may store various types of information, a computer program, a predetermined calculation formula for calculating the third physiological information, the correspondence relationship table T1, and the like. The storage device 26 is a storage device such as a hard disk drive (HDD), a solid state drive (SSD), or a flash memory.

In the above embodiments, an example has been described in which the weight information (that is, the second physiological information) is missing, the presently disclosed subject matter is also applicable to a case where the first physiological information and the second physiological information are missing or a case where the first physiological information is missing.

In the above embodiments, the display data may include at least one of the missing information, the missing physiological type information, and the uncalculated physiological type information, and may not include all of the missing information, the missing physiological type information, and the uncalculated physiological type information.

In the above embodiments, the controller 22 is set to calculate the continuous cardiac index and the pulmonary vascular resistance index, but may be set to calculate other third physiological information.

As described above, the present specification discloses items described in the following clauses.

### Clause 1

An information processing device including:
one or more processors; and
one or more memories configured to store at least one instruction configured to be executed by the one or more processors,
in which the information processing device is configured to acquire:
   first physiological information of a subject; and
   second physiological information of the subject, the second physiological information being different from the first physiological information,
the information processing device is configured to calculate third physiological information, based on the first physiological information, the second physiological information, and a predetermined calculation formula stored in advance in the one or more memories, and
when the at least one instruction is executed by the one or more processors,
   in a case where there is the first physiological information and/or the second physiological information that have not been acquired by the information processing device in calculating the third physiological information,
   the information processing device generates missing information indicating that there is the first physiological information and/or the second physiological information that have not been acquired by the information processing device among the first physiological information and the second physiological information necessary for calculating the third physiological information.

### Clause 2

The information processing device according to clause 1,
in which the information processing device is configured to receive the first physiological information and the second physiological information from an external device to acquire the first physiological information and the second physiological information, the external device being configured to communicate with the information processing device.

### Clause 3

The information processing device according to clause 1 or 2,
in which the information processing device is configured to output the missing information, and
the information processing device is configured to output the missing information a predetermined number of times, until the information processing device acquires confirmation information indicating that a user has confirmed the missing information after the information processing device outputs the missing information.

### Clause 4

The information processing device according to any one of clauses 1 to 3,
in which the information processing device is configured to output the missing information, and
in a case where a predetermined time has elapsed after the information processing device outputs the missing information and acquires confirmation information indicating that a user has confirmed the missing information,
   the information processing device is configured to output again the missing information, as it is determined that there is the first physiological information and/or the second physiological information that have not been acquired by the information processing device among the first physiological information and the second physiological information specified as the first physiological information and the second physiological information necessary for calculating the third physiological information.

### Clause 5

The information processing device according to any one of clauses 1 to 4,
in which the information processing device is configured to generate display data including the missing information.

### Clause 6

The information processing device according to any one of clauses 1 to 5,
in which the information processing device is configured to generate missing physiological type information indicating types of the missing first physiological information and/or second physiological information, based on the missing information, and
the information processing device is configured to generate display data including the missing physiological type information.

### Clause 7

The information processing device according to any one of clauses 1 to 6,
in which the information processing device is configured to generate uncalculated physiological type information indicating a type of the third physiological information that is not capable of being calculated due to absence of the first physiological information and/or the second physiological information, based on the missing information, and
the information processing device is configured to generate display data including the uncalculated physiological type information.

### Clause 8

The information processing device according to any one of clauses 1 to 7,
in which the information processing device is configured to determine a type of the acquired first physiological information, and
the information processing device is configured to specify the third physiological information to be calculated by the information processing device, according to the type of the first physiological information.

### Clause 9

An information processing method performed by an information processing device,
the information processing device being configured to acquire:
   first physiological information of a subject; and
   second physiological information of the subject, the second physiological information being different from the first physiological information, and
the information processing device being configured to calculate third physiological information, based on the first physiological information, the second physiological information, and a predetermined calculation formula stored in advance in the information processing device,
the information processing method including:
   a step of generating missing information indicating that there is the first physiological information and/or the second physiological information that have not been acquired by the information processing device among the first physiological information and the second physiological information necessary for calculating the third physiological information, in a case where there is the first physiological information and/or the second physiological information that have not been acquired by the information processing device in calculating the third physiological information.

### Clause 10

A non-transitory computer-readable medium storing a computer program including at least one instruction configured to be executed by one or more processors of an information processing device,
the information processing device being configured to acquire:
   first physiological information of a subject; and
   second physiological information of the subject, the second physiological information being different from the first physiological information, and
the information processing device being configured to calculate third physiological information, based on the first physiological information, the second physiological information, and a predetermined calculation formula stored in advance in the information processing device,
in which when the at least one instruction is executed by the one or more processors, the computer program causes the information processing device to generate missing information indicating that there is the first physiological information and/or the second physiological information that have not been acquired by the information processing device among the first physiological information and the second physiological information necessary for calculating the third physiological information, in a case where there is the first physiological information and/or the second physiological information that have not acquired by the information processing device in calculating the third physiological information.

What is claimed is:

1. An information processing device comprising:
one or more processors; and
one or more memories configured to store at least one instruction configured to be executed by the one or more processors,
wherein the information processing device is configured to acquire:
first physiological information of a subject; and
second physiological information of the subject, the second physiological information being different from the first physiological information,
the information processing device is configured to calculate third physiological information, based on the first physiological information, the second physiological information, and a predetermined calculation formula stored in advance in the one or more memories, and
when the at least one instruction is executed by the one or more processors,
in a case where there is the first physiological information and/or the second physiological information that have not been acquired by the information processing device in calculating the third physiological information, the information processing device generates missing information indicating that there is the first physiological information and/or the second physiological information that have not been acquired by the information processing device among the first physiological information and the second physiological information necessary for calculating the third physiological information.

2. The information processing device according to claim 1,
wherein the information processing device is configured to receive the first physiological information and the second physiological information from an external device to acquire the first physiological information and the second physiological information, the external device being configured to communicate with the information processing device.

3. The information processing device according to claim 1,
wherein the information processing device is configured to output the missing information, and
the information processing device is configured to output the missing information a predetermined number of times, until the information processing device acquires confirmation information indicating that a user has confirmed the missing information after the information processing device outputs the missing information.

4. The information processing device according to claim 1,
wherein the information processing device is configured to output the missing information, and
in a case where a predetermined time has elapsed after the information processing device outputs the missing information and acquires confirmation information indicating that a user has confirmed the missing information, the information processing device is configured to output again the missing information, as it is determined that there is the first physiological information and/or the second physiological information that have not been acquired by the information processing device among the first physiological information and the second physiological information specified as the first physiological information and the second physiological information necessary for calculating the third physiological information.

5. The information processing device according to claim 1,
wherein the information processing device is configured to generate display data including the missing information.

6. The information processing device according to claim 1,
wherein the information processing device is configured to generate missing physiological type information indicating types of the missing first physiological information and/or second physiological information, based on the missing information, and
the information processing device is configured to generate display data including the missing physiological type information.

7. The information processing device according to claim 1,
wherein the information processing device is configured to generate uncalculated physiological type information indicating a type of the third physiological information that is not capable of being calculated due to absence of the first physiological information and/or the second physiological information, based on the missing information, and
the information processing device is configured to generate display data including the uncalculated physiological type information.

8. The information processing device according to claim 1,
wherein the information processing device is configured to determine a type of the acquired first physiological information, and
the information processing device is configured to specify the third physiological information to be calculated by the information processing device, according to the type of the first physiological information.

9. An information processing method performed by an information processing device,
the information processing device being configured to acquire:
first physiological information of a subject; and
second physiological information of the subject, the second physiological information being different from the first physiological information, and
the information processing device being configured to calculate third physiological information, based on the first physiological information, the second physiological information, and a predetermined calculation formula stored in advance in the information processing device,
the information processing method comprising:
a step of generating missing information indicating that there is the first physiological information and/or the second physiological information that have not been acquired by the information processing device among the first physiological information and the second physiological information necessary for calculating the third physiological information, in a case where there is the first physiological information and/or the second physiological information that have not been acquired by the information processing device in calculating the third physiological information.

10. A non-transitory computer-readable medium storing a computer program including at least one instruction configured to be executed by one or more processors of an information processing device,
the information processing device being configured to acquire:
first physiological information of a subject; and
second physiological information of the subject, the second physiological information being different from the first physiological information, and
the information processing device being configured to calculate third physiological information, based on the first physiological information, the second physiological information, and a predetermined calculation formula stored in advance in the information processing device,
wherein when the at least one instruction is executed by the one or more processors, the computer program causes the information processing device to generate missing information indicating that there is the first physiological information and/or the second physiological information that have not been acquired by the information processing device among the first physiological information and the second physiological information necessary for calculating the third physiological information, in a case where there is the first physiological information and/or the second physiological information that have not acquired by the information processing device in calculating the third physiological information.
